(19) United States
(12) Patent Application Publication
(10) Pub. No.: US 2026/0157809 A1
(43) Pub. Date: Jun. 11, 2026

(54) ADAPTOR FOR SURGICAL TOOL

(71) Applicant: PELETON SURGICAL, LLC, SCOTTSDALE, AZ (US)

(72) Inventor: David John Reed, Sheffield (GB)

(21) Appl. No.: 19/411,648

(22) Filed: Dec. 8, 2025

Related U.S. Application Data
(60) Provisional application No. 63/728,830, filed on Dec. 6, 2024.

(51) Int. Cl. A61B 34/30; A61B 17/00; A61B 17/14; A61B 17/16; A61B 17/88; A61B 90/00

(52) U.S. Cl. CPC A61B 34/30; A61B 17/14; A61B 17/1626; A61B 17/8872; A61B 90/37; A61B 2017/00022; A61B 2017/00199; A61B 2017/00225; A61B 2017/0023; A61B 2017/00367; A61B 2017/00477

(57) ABSTRACT

A system for a surgical procedure is provided. A robotic system includes a robotic arm. A surgical tool includes a driver and an attachment. The sterile, single-use driver is operable to provide power to and manipulate the attachment. An adaptor includes a housing operable to be detachably coupled with the surgical tool. The adaptor includes a robot coupling component operable to be detachably coupled with the robotic arm. When the adaptor is coupled with the robotic arm, the robotic arm is operable to manipulate a position and/or movement of the surgical tool. When the adaptor is detached from the robotic arm, the surgical tool is operable to be manually manipulated.

### CROSS-REFERENCE TO RELATED APPLICATION

This application claims the benefit of U.S. Provisional Patent Application No. 63/728,830, filed in the U.S. Patent and Trademark Office on Dec. 6, 2024, which is incorporated herein by reference in its entirety for all purposes.

### TECHNICAL FIELD

The present disclosure generally relates to a power tool and a method of supplying power to a power tool, and more particularly to a portable device powered by a chargeable battery and a method of supplying power to the portable device powered by a chargeable battery.

### BACKGROUND

Medical equipment and instruments are required to maintain an aseptic condition in order to protect the safety and health of patients. While mobile power supplies are available, existing power supply devices and methods of supplying power are not able to charge medical equipment and instruments located in a sealed and sterile environment. Rather, these power supplies and methods thereof are surrounded by dust and bacteria, and are susceptible to contact by harmful fluids. Accordingly, the sterility and safety of using these power supplies to charge electrical medical equipment and instruments is degraded and the service life is significantly reduced. Additionally, the patient's health can be compromised.

### BRIEF DESCRIPTION OF THE DRAWINGS

In order to describe the manner in which the above-recited and other advantages and features of the disclosure can be obtained, a more particular description of the principles briefly described above will be rendered by reference to specific embodiments thereof, which are illustrated in the appended drawings. Understand that these drawings depict only exemplary embodiments of the disclosure and are not, therefore, to be considered to be limiting of its scope, the principles herein are described and explained with additional specificity and detail through the use of the accompanying drawings.

FIG. 1A illustrates a surgical environment of using the system with the surgical tool coupled with the robotic system via the adaptor.

FIG. 1B illustrates the surgical tool and the adaptor detached from the robotic system and in use.

FIG. 2A illustrates the surgical tool.

FIG. 2B illustrates a schematic view of the driver of the surgical tool.

FIG. 3A illustrates an example of the driver in a tubular configuration.

FIG. 3B illustrates a schematic view of the driver of FIG. 3A.

FIG. 4 illustrates the surgical tool provided in a packaging.

FIG. 5 illustrates the surgical tool coupled with the adaptor.

FIG. 6 is a schematic diagram of a controller which may be employed as shown in FIGS. 1A-5.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without departing from the spirit and scope of the disclosure. Additional features and advantages of the disclosure will be outlined in the description which follows, and in part will be obvious from the description, or can be learned by practice of the herein disclosed principles. It will be appreciated that for simplicity and clarity of illustration, where appropriate, reference numerals have been repeated among the different figures to indicate corresponding or analogous elements. The description is not to be considered as limiting the scope of the embodiments described herein.

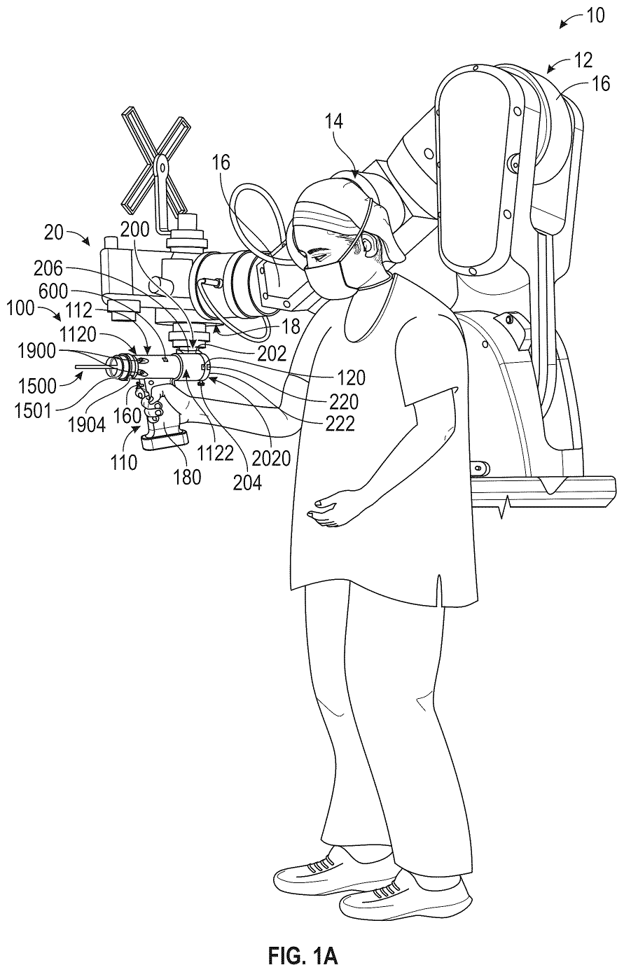

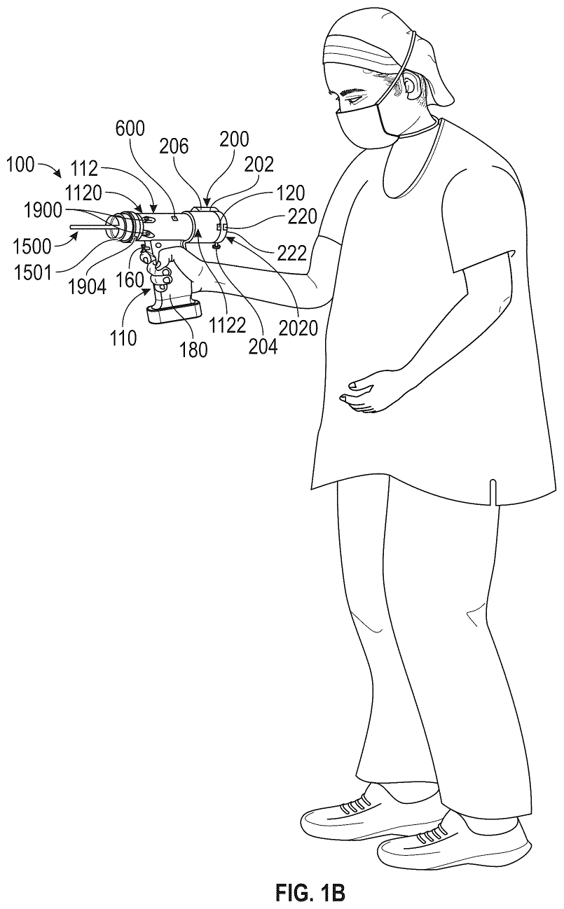

Referring to FIGS. 1A and 1B, a system 10 is used in a surgical environment. The system 10 can include a surgical tool 100. The surgical tool 100 can include a driver 110 and one or more attachments 1500.

The system 10 can include a robotic system 12. In at least one example, the robotic system 12 can include a multi-arm surgical robot, a single-port robot, an orthopedic robotic arm, a neurosurgical positioning robot, etc. For example, as illustrated in FIG. 1A, the robotic system 12 can include a robotic arm 14 that can have one or more articulated joints 16 to move the end 18 of the robotic arm 14. The end 18 of the robotic arm 14 can be operable to be detachably coupled with the surgical tool 100 via an adaptor 200 that is coupled with the surgical tool 100, as will be discussed in more detail below.

In some examples, as illustrated in FIG. 1A, the robotic system 12 can include one or more imaging devices 20. For example, the imaging devices 20 can include one or more cameras, such as 2-dimensional cameras, 3-dimensional cameras, CT, MRI, etc. that are operable to capture images during the surgical procedure. A controller (not shown) for the robotic system 12 can be operable to receive the images from the imaging devices 20. The controller for the robotic system 12 can then perform an analysis of the images. For example, the controller may utilize machine learning techniques and training to determine the positioning and/or the movement of the surgical tool 100, and determine whether the positioning and/or movement of the surgical tool 100 is correct for the step of the surgical procedure. The controller can then manipulate the position and/or movement of the surgical tool 100 based on the analysis of the images to assist with the surgical procedure. For example, the controller may manipulate the robotic arm 14 to control the positioning and/or movement of the surgical tool 100. In some examples, the controller may manipulate the surgical tool 100 (e.g., the driver 110) to control the power and/or control of the attachment 1500. In such examples, the robotic system 12 can be communicatively coupled with the surgical tool 100, for example by wired or wireless connection, and the controller of the robotic system 12 can provide signals to the controller 600 of the surgical tool 100. In response, the controller 600 of the surgical tool 100 can provide feedback based on sensor data (for example from force sensor 1904), so that the robotic system 12 can manipulate the robotic arm 14 accordingly.

In some examples, the controller may manipulate the robotic arm 14 to control the positioning and/or movement of the surgical tool 100 in conjunction with the operator (for example as shown in FIG. 1A) manipulating the control of the surgical tool 100 (e.g., the driver 110). In such an example, the operator can maintain control of the procedure overall and use their vision, feel, and knowledge to ensure that the surgical tool 100 is correctly interacting with the patient during the surgical procedure. Meanwhile, the robotic system 12, such as the robotic arm 14 and the imaging devices 20, can assist the operator to ensure that the correct portions are being operated on (e.g., being cut). This harmonization between the robotic system 12 and the operator can improve the surgical procedure and its results.

As shown in FIG. 1B, the surgical tool 100 with the adaptor 200 can easily be detached from the robotic system 12, allowing for the surgical tool 100 to be manually manipulated by the operator. This allows for the operator to quickly remove the surgical tool 100 from the robotic system 12 and have more dexterity and control over the surgical tool 100, and subsequently the surgical procedure. The ability provided by the adaptor 200 to quickly and easily attach and detach the surgical tool 100 from the robotic system 12 provides for the flexibility, control, and assistance where needed, for the operator to successfully complete the surgical procedure.

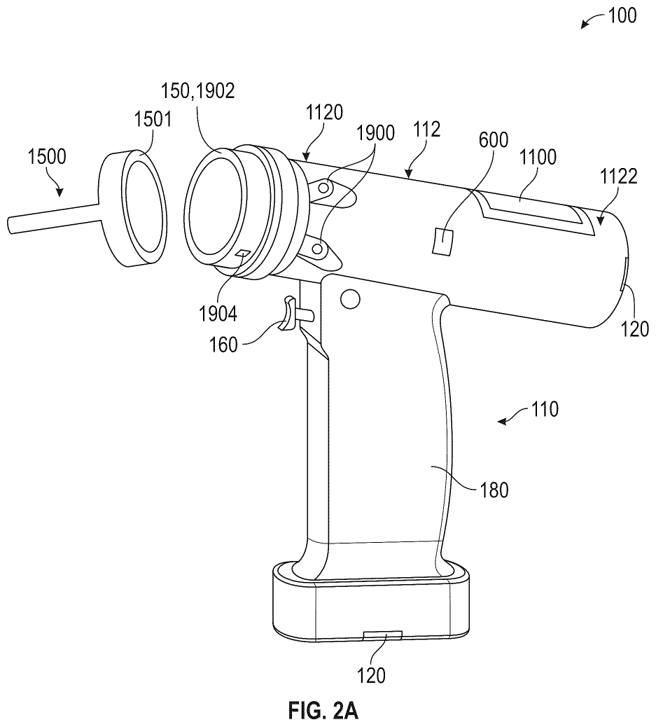

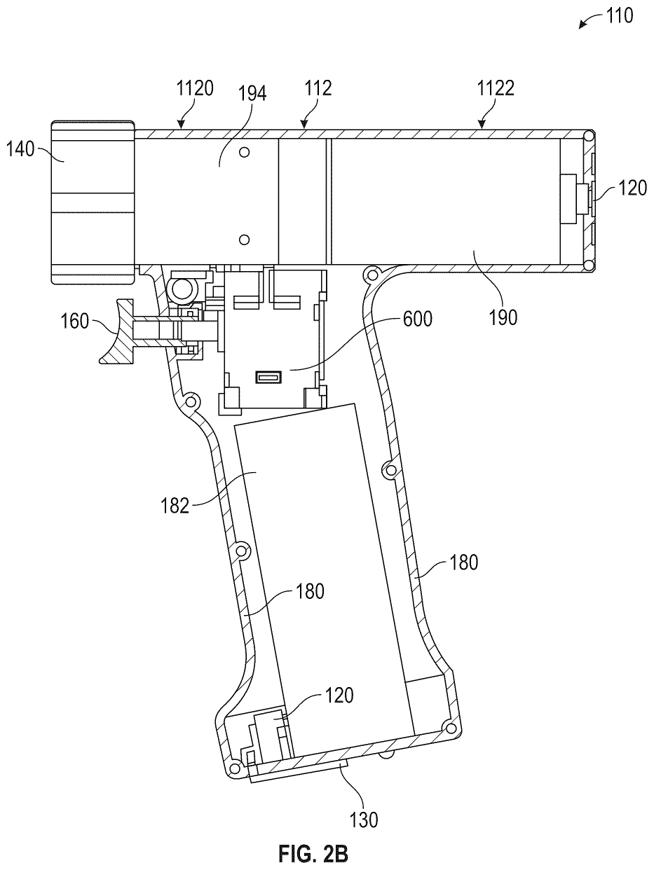

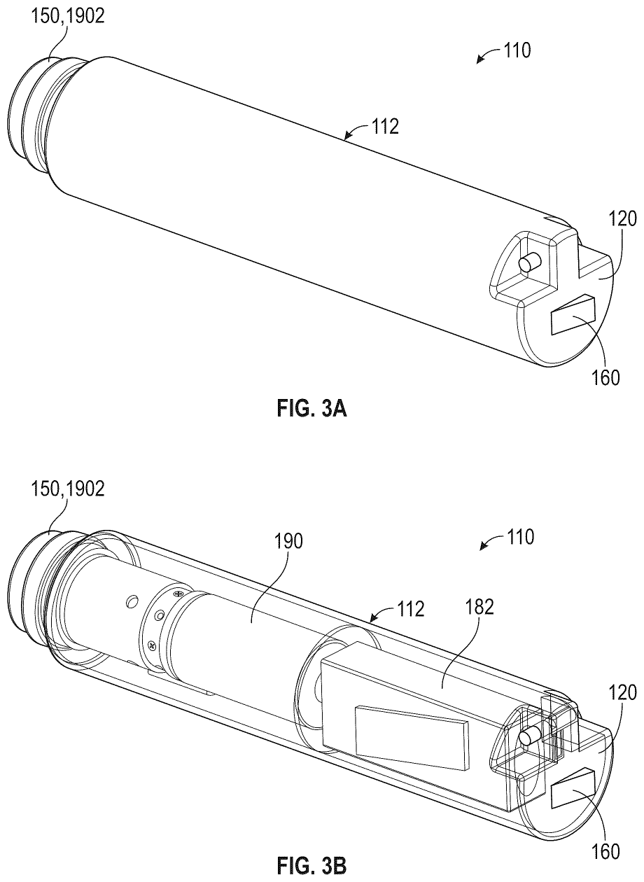

FIGS. 2A, 2B, 3A, and 3B illustrate the surgical tool 100. In FIG. 2A, the driver 110 is illustrated with an attachment 1500. For example, the surgical tool 100 can include a driver 110 which can be operable to provide power to and manipulate the attachment(s) 1500. It should be appreciated that an attachment 1500 may be selected from a plurality of attachment types and may include, but are not limited to, saw blades, wire/pin drivers, and/or drill chucks.

In at least one example, the attachment 1500 can be operable to be coupled with the driver 110 via coupling mechanism 150. The coupling mechanism 150 can include a device coupling 1902 and a corresponding attachment coupling 1501. In some examples, the device coupling 1902 and the attachment coupling 1501 can be operable to couple with each other via threaded engagement, snap fit, etc. In some examples, the device coupling 1902 and the attachment coupling 1501 can be operable to couple with each other via magnetic attachment. In some examples, the device coupling 1902 can include magnet(s) while the attachment coupling 1501 can include a ferromagnetic material. In some examples, the attachment coupling 1501 can include magnet(s) while the device coupling 1902 can include a ferromagnetic material. In some examples, both the attachment coupling 1501 and the device coupling 1902 can include magnet(s). The magnetic attachment mechanism can also assist in aligning the attachment 1500 onto the driver 110 to make sure that the correct placement, positioning, and/or alignment of the attachment 1500 is provided.

It should be appreciated that a spring-loaded collar may be included in surgical tool 100 and may engage an attachment. It should further be appreciated that a spring-loaded collar may be pulled backwards along a central axis of driver 110, and when the spring-loaded collar is released, it may spring forward and securely hold the attachment in place. It should also be appreciated that an attachment may automatically engage with internal drive shaft 194 (FIG. 2B). It should be appreciated that an attachment may be removed from attachment coupling 150 by pulling a spring-loaded collar backwards along a central axis of driver 110, and may provide for easily removing the attachment. Trigger 160 may be provided to vary the speed of rotation of the interior of attachment coupling. It should be appreciated that trigger 160 may be provided to control the direction of rotation of the interior of attachment coupling 150 in a clockwise direction or in a counterclockwise direction without departing from the present disclosure. It should be appreciated that driver 110 may provide a variable-speed trigger and an instant-reverse trigger in some embodiments of the present disclosure. Driver 110 may also provide at least one grip 180 extending from the body 112 that may stabilize driver 110 in the user's hands without departing from the present disclosure. In at least one example, the grip 180 can extend from the body 112 substantially transverse from the longitudinal axis of the body 112. It should be appreciated that the at least one grip 180 may be textured.

The driver 110 may provide battery 182. Battery 182 may be arranged inside of driver 110 and may be charged by connecting a cable to port 120. Driver 110 may include motor 190 and controller 600. Motor 190 may control the speed of attachments that may be provided inside of attachment coupling 150. Controller 600 may provide the electrical components required to operate at least trigger 160, motor 190, and attachment coupling 150. Internal drive shaft 194 may be connected to attachment coupling 150 and provide for an engagement of surgical tool 100 with an attachment 1500.

As shown in FIGS. 2A and 2B, the driver 110 may provide one or more ports 120 and, in some examples, corresponding port cover(s) 130. The port 120 may include a female port that may be provided to receive a connector (for example, a cable, a link connector 220, etc.), and the port 120 may be covered and protected by port cover 130. It should be appreciated that a female port may include, but is not limited to, a female USB port, a female cord end, and/or female wire end without departing from the present disclosure. It should be appreciated that port cover 130 may slide between a locked position and unlocked position in some examples of the present disclosure. It should be appreciated that the locked position may prevent connectors or another cable from attaching to charging port 120.

In some examples, the port 120 can be operable to receive power for the surgical tool 100. In some examples, the port 120 can be operable to receive and/or transmit signals, for example signals to and/or from the robotic system 12. In some examples, the driver 110 can be operable to receive instructions via the signals received through the port 120 to operate the driver 110. In some examples, a single port 120 can be operable to both receive power for the surgical tool 100 as well as receive and/or transmit signals. Accordingly, less ports 120 would be needed.

In some examples, as illustrated in FIGS. 2A and 2B, the port 120 can be positioned on the grip 180 of the driver 110. In some examples, the port 120 can be positioned at a rear end of the body 112 of the driver 110. For example, as illustrated in FIGS. 2A and 2B, the body 112 can include a front portion 1120 that is between the attachment 1500 and the grip 180, and a rear portion 1122 that is opposite the front portion in relation to the grip 180. The port 120 can be positioned on the rear portion 1122 of the body 112.

In at least one example, the driver 110 can include one or more light emitters 1900. In some examples, the light emitters 1900 can be operable to illuminate the operative field when the tools were being used. In some examples, the light emitters 1900 can be operable to shine deep into the surgical site and provide extra visibility for the doctor during whatever procedure they are performing.

In some examples, the light emitters 1900 can include a focused light beam (e.g., laser) that can be directed down at the tool and/or attachment orientation. With the focused light beam, the driver 110 can provide angular orientation as feedback to the surgeon during operation (e.g., drilling and/or sawing).

In some examples, the light emitters 1900 may emit light to signal whether the attachment 1500 is correctly coupled with the driver 110. For example, the light emitters 1900 may flash light a predetermined number of times (e.g., 2, 3, 4, etc.) when the attachment 1500 is correctly coupled with the driver 110. This can confirm the connection is fully engaged and the surgical tool 100 is ready to be utilized.

In at least one example, the device display 1100 can be operable to provide a digital readout of the rotations per minute speed, depending on how far the trigger is depressed. In some examples, the device display 1100 can be operable to display the level of torque being applied at any stage of the procedure, or other information such as operating temperature could be shown.

In some examples, as illustrated in FIGS. 2A and 2B, the driver 110 can include a force sensor 1904. The force sensor 1904 can be operable to monitor the force being applied during any particular procedure and, in some examples, can cut off power automatically if required to prevent unnecessary tissue damage. For example, when the surgical tool 100 is being used to cut or drill thru a bone, as soon as the force sensor 1904 detects a reduction in pressure the power can be automatically cut off to disable the function until the user presses the trigger again to reset. In some examples, the measured reduction in pressure by the force sensor 1904 can be utilized by the controller 600 to signal an alert to inform the operator that there was a reduction in pressure. In some examples, the measured reduction in pressure by the force sensor 1904 can be transmitted by the controller 600 to the robotic system 12 so that the robotic system 12 adjusts the manipulation of the surgical tool 100, for example by stopping movement of the robotic arm 14 and correspondingly the surgical tool 100 so that the surgical tool 100 does not move and potentially cut something undesired.

Any of the features discussed and illustrated in FIGS. 2A and 2B can be implemented along with any of the features discussed and illustrated in FIGS. 3A and 3B. FIGS. 3A and 3B illustrate that the body 112 of the driver 110 can be substantially linear, such as being substantially tubular, without a grip 180 extending therefrom. In such examples, the robotic system 12 may have full control over the surgical tool 100 when the adaptor 200 and the surgical tool 100 are coupled with the robotic system 12.

It should be appreciated that embodiments of the present disclosure may provide for usage in conflict zones or natural disasters, where charging equipment may not be possible. Further, usage may be provided in geographic locations that may not have access to an adequate power supply and/or sterile environment cannot fully charge medical equipment and instruments. However, usage in hospitals and other medical facilities may be improved insofar as devices containing tools necessary for medical procedures may be readily available off-the-shelf and for immediate use at a cost that has significant advantages over other reusable tool systems.

The surgical tool 100 is importantly configured to be sterile, single-use, and disposable. Accordingly, the surgical tool 100 can be ensured to be sterile and ready to be used directly out of the sterile packaging. This can save the operating team much time in cleaning, sterilizing, and charging the surgical tool 100. Accordingly, the surgical tool 100 can be utilized quickly and also in areas that may not have sufficient amenities and support.

It should be appreciated that surgical tool 100 may be a battery-driven tool system that may be used for medical procedures including, but not limited to, drilling, reaming, pin and wire placement, and cutting bone and hard tissue. It should be appreciated that surgical tool 100 may be operated for non-medical use including, but not limited to, construction, household-use, and food preparation. It should be appreciated that surgical tool 100 may provide power for immediate use after opening the package 50. It should be appreciated that surgical tool 100 may provide cost advantages over reusable portable devices. It should further be appreciated that a portable device according to examples of the present disclosure may be used one time and may be recycled and/or discarded after use. It should be appreciated that surgical tool 100 may eliminate a need for maintenance and lubrication. It should also be appreciated that a portable device according to embodiments of the present disclosure may eliminate a need for back-up batteries and/or a back-up power supply. It should further be appreciated that surgical tool 100 may not require special processes for cleaning and/or disposal of any component.

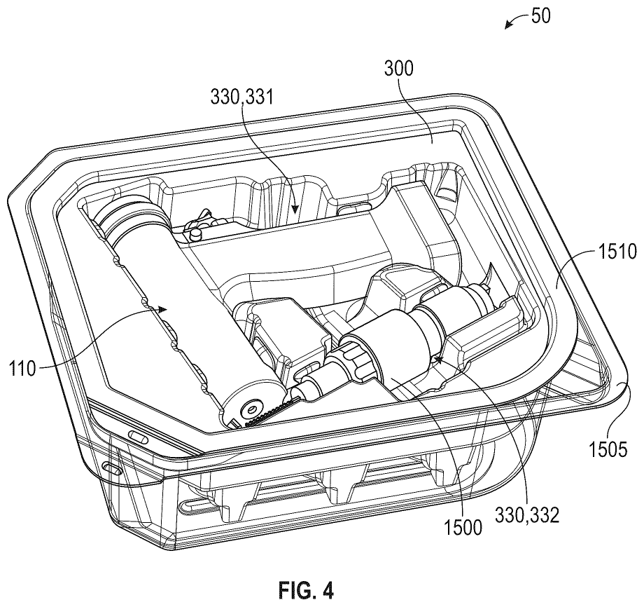

FIG. 4 illustrates an example of the driver 110 of the surgical tool 100 being secured within a tray 300 along with one or more attachments 1500. For example, the surgical tool 100 can include a driver 110 which can be operable to provide power to and manipulate the attachments 1500. It should be appreciated that an attachment may be selected from a plurality of attachment types and operable to be inserted into attachment coupling 150. The attachments 1500 may include, but are not limited to, saw blades, wire/pin drivers, and/or drill chucks. The tray 300 can include a first compartment 331 operable to securely receive the driver 110 of the surgical tool 100. The tray 300 can also include a second compartment 332 operable to securely receive an attachment 1500. Accordingly, a kit can be provided that includes all the necessary tools (e.g., driver 110 and attachments 1500) to perform the procedure.

In some examples, not shown here, the tray 300 can be operable to receive and secure the driver 110 of the surgical tool 100 while a second tray can be operable to receive and secure the tray 300. The package 50 can be operable to receive tray 300 and the second tray in a nested configuration to provide a sterile enclosure for the surgical tool 100.

It should be appreciated that package 50 may create a sterile barrier system (SBS). It should be appreciated that the SBS may prevent an ingress of microorganisms from reaching surgical tool 100, but may allow the passage of air and sterilizing media to contact surgical tool 100. Sterilizing media may include, but is not limited to, ethylene oxide (ETO), steam, gamma irradiation, and electron beam (eBeam), and may help to maintain a sterile environment for surgical tool 100 prior to use. It should be appreciated that the sterile environment may provide sterile asepsis to eliminate micro-organisms from the portable device. It should further be appreciated that package 50, including tray 300 and second tray, may be made of material including, but not limited to, paper, laminated film, plastic, and foil that may provide a sterile barrier.

It should be appreciated that package 50 may form a second packaging that may facilitate safe storage and handling of surgical tool 100. It should be appreciated that package 50 may contain any number of trays or primary packages without departing from the present disclosure.

In some examples, more than one surgical tool 100 can be received in a single first tray which is then received in a second tray. The package 50, along with any configuration of portable devices 100, first tray(s), and/or second tray(s), provides a sterile environment to charge the portable devices 100 either individually, together all at the same time, or any combination of portable devices 100. For example, the power supply can provide a charge to all of the portable devices until all of the portable devices are fully charged or charged to the desired amount. In some examples, the power supply can provide a charge to the portable devices as needed for each individual portable device.

To utilize the surgical tool 100, such as the driver 110, the outer second tray can be firstly opened within the sterile area (e.g., the operating room). The inner tray 300 can then be removed from the second tray. The cover of the inner tray 300 can be peeled back (e.g., by the scrub nurse) to reveal the driver 110, which is then presented to the doctor aseptically to remove the driver 110 from the tray 300. Accordingly, to ensure the sterile environment and that the surgical tool 100 is sterile, at least two sterile levels (e.g., the tray 300 and the second tray) is necessary to deliver a sterile device. While the disclosure discusses two sterile levels in the tray 300 and the second tray, more than two sterile levels can be included as more trays can be nested together. Conventional systems include only one sterile layer, which defeats the objective of the sterilization process because the device is compromised as soon as the cover of the single layer is opened.

In some examples, surgical tool 100 may be charged while inside of package 50 by connecting power supply (not shown) to the driver 110 within the package 50. Accordingly, the surgical tool 100 can be charged while remaining within the sterile environment, and the surgical tool 100 can be used right out of the packaging 50 by the operator, without the need of additional wait time in a sterile room for charging. That additional wait time could lead to more chances of losing sterility of the surgical tool 100, and could waste time in an urgent procedure.

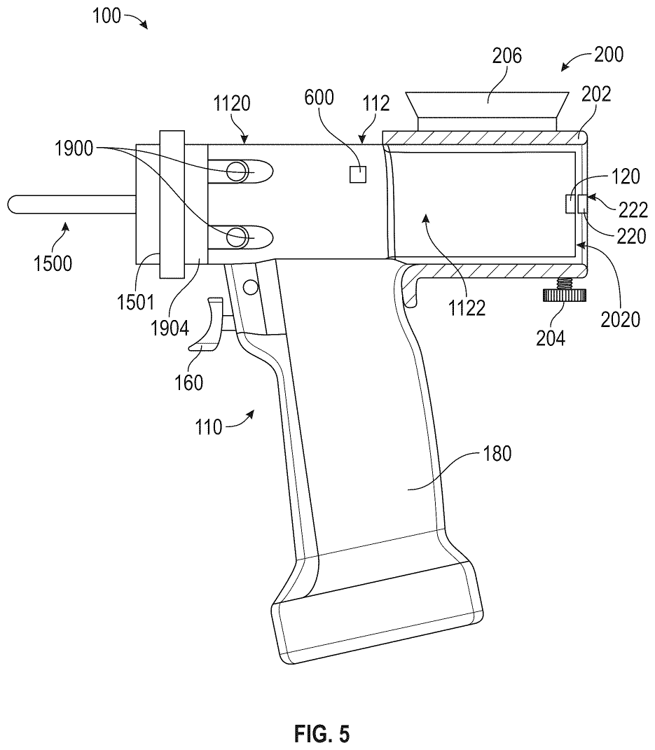

As shown in FIGS. 1A, 1B, and 5, the surgical tool 100 can be detachably coupled with the adaptor 200 which provides a mechanism for the surgical tool 100 to be detachably coupled with the robotic system 12 (e.g., the robotic arm 14). Importantly, the adaptor 200 and the surgical tool 100 are both configured to be sterile, single-use, and disposable. Accordingly, similar to the surgical tool 100, the adaptor 200 does not need to be sterilized, and can be used straight out of the packaging.

The adaptor 200 includes a housing 202 that is operable to be detachably coupled with the surgical tool 100. For example, as shown in FIGS. 1A, 1B, and 5, the adaptor 200 can be configured to be coupled with the rear portion 1122 of the body 112. Accordingly, when the adaptor 200 is coupled with the surgical tool 100, the attachments 1500 can be easily attached and detached without the adaptor 200 interfering. Also, the adaptor 200 can be coupled to the surgical tool 100 without interference or needing to remove the attachments 1500. Additionally, the adaptor 200 can be easily coupled with the surgical tool 100 without interference from the grip 180.

In at least one example, the housing 202 of the adaptor 200 can have a substantially tubular shape forming a lumen 2020. The lumen 2020 can be operable to receive at least a portion (e.g., the rear portion 1122) of the body 112 of the driver 110. Accordingly, the adaptor 200 can easily be slid on to the rear portion 1122 of the body 112 of the driver 110, to allow for efficient and simple coupling.

In some examples, the adaptor 200 can be detachably coupled with the surgical tool 100 via a tool coupling component 204. In some examples, the tool coupling component 204 can include includes a thumb screw, a snap fit, a clamp, a quick-release lever, a slide, a set screw, a magnetic mount, and/or a kinematic ball coupling.

The adaptor 200 includes a robot coupling component 26 that is operable to be detachably coupled with the robotic system 12 (e.g., the robotic arm 14). Accordingly, the adaptor 200 allows for the surgical tool 100 to be detachably coupled with the robotic system 12. As the adaptor 200 allows for easy and simple detaching of the surgical tool 100 from the robotic system 12, the adaptor 200 allows for quick and efficient switch from the operator working with the robotic system 12 to the operator taking full control with manual manipulation of the surgical tool 100. This allows for the flexibility needed during the quick-moving surgical procedure. In at least one example, the robot coupling component 206 can include a dovetail slide, one or more magnets, a lever, a collet connector, a chuck connector, a twist-lock interface, an electromagnetic retention mechanism, a keyed mechanical mount, a threaded coupling, and/or a latch clamp mechanism. Accordingly, with the adaptor 200, the surgical tool 100 can quickly and easily be coupled with the robotic system 12 allowing for the desired control and manipulation of the surgical tool 100, and quickly and easily be detached from the robotic system 12 allowing for manual manipulation by the operator.

In at least one example, the adaptor 200 can include a link connector 220 that is operable to connect the port 120 of the surgical tool 100 with the robotic system 12 and/or a conduit (e.g., data and/or power transmission).

In some examples, the link connector 220 can be operable to be received by the port 120 when the adaptor 200 is coupled with the surgical tool 100. For example, as the housing 202 of the adaptor 200 is slid and/or placed on the rear portion 1122 of the driver 110, the link connector 220 can be received by and couple with the port 120 that is provided on the rear portion 1122 of the body 112. Accordingly, there is also feedback and confirmation of sufficient connection between the adaptor 200 and the driver 110 when the link connector 220 is received by the port 120. The link connector 220 can then serve as a pass through connection for power and/or signal transmission. The link connector 220 can then be coupled with a power cable, a transmission cable, and/or the robotic system 12. In some examples, the link connector 220 can provide wireless capabilities to the surgical tool 100 and connect to systems such as the robotic system 12. With the link connector 220 coupling with the port 120 on the rear portion 1122 of the driver 110, the adaptor 200 provides a simple connection without changing the function of the surgical tool 100. Accordingly, when the surgical tool 100 with the adaptor 200 is quickly attached or detached from the robotic system 12 during the surgical procedure, there is no interference or pause with the surgical procedure to move connections around.

In some examples, when the port 120 is provided on the grip 180 of the driver 110, a connection cable, such as a power and/or a transmission cable, can be continually connected even when the surgical tool 100 is attached and detached from the robotic system 12. The connection can then remain secure without interference from connecting the adaptor 200.

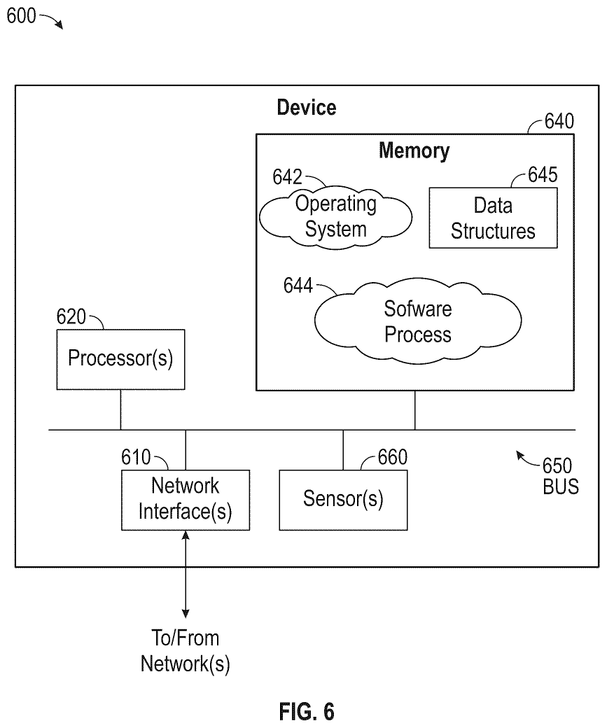

FIG. 6 is a block diagram of an exemplary controller 600. Controller 600 is configured to perform processing of data and communicate, for example as illustrated in FIGS. 1A-5. In operation, controller 600 communicates with one or more of the components discussed herein and may also be configured to communication with remote devices/systems.

As shown, controller 600 includes hardware and software components such as network interfaces 610, at least one processor 620, sensors 660 and a memory 640 interconnected by a system bus 650. Network interface(s) 610 can include mechanical, electrical, and signaling circuitry for communicating data over communication links, which may include wired or wireless communication links. Network interfaces 610 are configured to transmit and/or receive data using a variety of different communication protocols, as will be understood by those skilled in the art.

Processor 620 represents a digital signal processor (e.g., a microprocessor, a microcontroller, or a fixed-logic processor, etc.) configured to execute instructions or logic to perform tasks in a medical and/or surgical environment. Processor 620 may include a general purpose processor, special-purpose processor (where software instructions are incorporated into the processor), a state machine, application specific integrated circuit (ASIC), a programmable gate array (PGA) including a field PGA, an individual component, a distributed group of processors, and the like. Processor 620 typically operates in conjunction with shared or dedicated hardware, including but not limited to, hardware capable of executing software and hardware. For example, processor 620 may include elements or logic adapted to execute software programs and manipulate data structures 645, which may reside in memory 640.

Sensors 660 typically operate in conjunction with processor 620 to perform measurements, and can include special-purpose processors, detectors, transmitters, receivers, and the like. In this fashion, sensors 660 may include hardware/software for generating, transmitting, receiving, detection, logging, and/or sampling parameters.

Memory 640 comprises a plurality of storage locations that are addressable by processor 620 for storing software programs and data structures 645 associated with the embodiments described herein. An operating system 642, portions of which may be typically resident in memory 640 and executed by processor 620, functionally organizes the device by, inter alia, invoking operations in support of software processes and/or services 644 executing on controller 600. These software processes and/or services 644 may perform processing of data and communication with controller 600, as described herein. Note that while process/service 644 is shown in centralized memory 640, some examples provide for these processes/services to be operated in a distributed computing network.

It will be apparent to those skilled in the art that other processor and memory types, including various computer-readable media, may be used to store and execute program instructions pertaining to the techniques described herein. Also, while the description illustrates various processes, it is expressly contemplated that various processes may be embodied as modules having portions of the process/service 644 encoded thereon. In this fashion, the program modules may be encoded in one or more tangible computer readable storage media for execution, such as with fixed logic or programmable logic (e.g., software/computer instructions executed by a processor, and any processor may be a programmable processor, programmable digital logic such as field programmable gate arrays or an ASIC that comprises fixed digital logic. In general, any process logic may be embodied in processor 620 or computer readable medium encoded with instructions for execution by processor 620 that, when executed by the processor, are operable to cause the processor to perform the functions described herein.

Although the present disclosure and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, and composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A system for a surgical procedure, the system comprising:
a robotic system including a robotic arm; a surgical tool including a driver and an attachment, wherein the driver is operable to provide power to and manipulate the attachment; and
an adaptor including a housing operable to be detachably coupled with the surgical tool, wherein the adaptor includes a robot coupling component operable to be detachably coupled with the robotic arm,
wherein when the adaptor is coupled with the robotic arm, the robotic arm is operable to manipulate a position and/or movement of the surgical tool,
wherein when the adaptor is detached from the robotic arm, the surgical tool is operable to be manually manipulated.

2. The system of claim 1, wherein the adaptor and the surgical tool are both configured to be sterile, single-use, and disposable.

3. The system of claim 1, wherein the robotic system includes one or more imaging devices to capture images during the surgical procedure, wherein the robotic system includes a controller that is operable to receive the images and manipulate the position and/or movement of the surgical tool based on an analysis of the images to assist with the surgical procedure.

4. The system of claim 1, wherein the surgical tool includes a port, wherein the port is operable to receive power for the surgical tool.

5. The system of claim 4, wherein the port is operable to receive and/or transmit signals.

6. The system of claim 5, wherein the driver is operable to receive instructions via the signals to operate the driver.

7. The system of claim 4, wherein the adaptor includes a link connector that is operable to connect the port of the surgical tool with the robotic system and/or a conduit.

8. The system of claim 7, wherein the link connector is operable to be received by the port when the adaptor is coupled with the surgical tool.

9. The system of claim 4, wherein the port is positioned on a grip of the driver.

10. The system of claim 4, wherein the port is positioned at a rear end of a body of the driver.

11. The system of claim 1, wherein the driver includes a body and a grip extending from the body, wherein the body includes a front portion that is between the attachment and the grip, and a rear portion that is opposite the front portion in relation to the grip,
wherein the adaptor is configured to be coupled with the rear portion of the body.

12. The system of claim 1, wherein the housing of the adaptor has a substantially tubular shape forming a lumen, wherein the lumen is operable to receive at least a portion of a body of the driver.

13. The system of claim 1, wherein the adaptor is detachably coupled with the surgical tool via a tool coupling component.

14. The system of claim 13, wherein the tool coupling component includes a thumb screw, a snap fit, a clamp, a quick-release lever, a slide, a set screw, a magnetic mount, and/or a kinematic ball coupling.

15. The system of claim 1, wherein the robot coupling component includes a dovetail slide, one or more magnets, a lever, a collet connector, a chuck connector, a twist-lock interface, an electromagnetic retention mechanism, a keyed mechanical mount, a threaded coupling, and/or a latch clamp mechanism.

16. The system of claim 1, wherein the attachment is detachably coupled with the driver.

17. The system of claim 1, wherein the attachment includes a saw blade, a wire/pin driver, and/or a drill chuck.

18. An adaptor for a surgical tool, the adaptor comprising:
a housing operable to be detachably coupled with the surgical tool; and
a robot coupling component operable to be detachably coupled with a robotic system, wherein when the adaptor is coupled with the robotic system, the robotic system is operable to manipulate a position and/or movement of the surgical tool,
wherein when the adaptor is detached from the robotic system, the surgical tool is operable to be manually manipulated.

19. The adaptor of claim 18, wherein the adaptor is configured to be sterile, single-use, and disposable.

20. The adaptor of claim 18, further comprising a link connector that is operable to connect a port of the surgical tool with the robotic system and/or a conduit.
